(19) United States
(12) Patent Application Publication
(10) Pub. No.: US 2024/0140976 A1
(43) Pub. Date: May 2, 2024

(54) PROCESS FOR PREPARING AN E-SELECTIN INHIBITOR INTERMEDIATE

(71) Applicant: GLYCOMIMETICS, INC., Rockville, MD (US)

(72) Inventor: Indranath GHOSH, Olney, MD (US)

(73) Assignee: GLYCOMIMETICS, INC., Rockville, MD (US)

(21) Appl. No.: 18/546,609

(22) PCT Filed: Feb. 17, 2022
(86) PCT No.: PCT/US2022/016696

§ 371 Date: Aug. 16, 2023

Related U.S. Application Data
(60) Provisional application No. 63/150,940, filed on Feb. 18, 2021.

(51) Int. Cl. C07H 15/207

(52) U.S. Cl. CPC C07H 15/207

(57) ABSTRACT

A process is provided for the synthesis of an intermediate which is useful in the synthesis of E-selectin inhibitors. Also provided are useful intermediates obtained from the process.

This application claims the benefit under 35 U.S.C. § 119(e) to U.S. Provisional Application No. 63/150,940 filed Feb. 18, 2021, which application is incorporated by reference herein in its entirety.

A process is provided for the synthesis of an intermediate which is useful in the synthesis of E-selectin inhibitors. Also provided are useful intermediates obtained from the process. This class of compounds is described in, for example, U.S. Pat. Nos. 9,796,745 and 9,867,841, U.S. patent application Ser. Nos. 15/025,730, 15/531,951, 16/081,275, 16/323,685, and 16/303,852, and PCT International Application No PCT/US2018/067961.

Selectins are a group of structurally similar cell surface receptors important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic domain spanning region and a cytoplasmic domain. The binding interactions appear to be mediated by contact of the lectin domain of the selectins and various carbohydrate ligands.

There are three known selectins: E-selectin, P-selectin, and L-selectin. E-selectin is found on the surface of activated endothelial cells, which line the interior wall of capillaries. E-selectin binds to the carbohydrate sialyl-Lewis^{x} (sLe^{x}), which is presented as a glycoprotein or glycolipid on the surface of certain leukocytes (monocytes and neutrophils) and helps these cells adhere to capillary walls in areas where surrounding tissue is infected or damaged; and E-selectin also binds to sialyl-Lewis^{a} (sLe^{a}), which is expressed on many tumor cells. P-selectin is expressed on inflamed endothelium and platelets, and also recognizes sLe^{x} and sLe^{a}, but also contains a second site that interacts with sulfated tyrosine. The expression of E-selectin and P-selectin is generally increased when the tissue adjacent to a capillary is infected or damaged. L-selectin is expressed on leukocytes. Selectin-mediated intercellular adhesion is an example of a selectin-mediated function.

Although selectin-mediated cell adhesion is required for fighting infection and destroying foreign material, there are situations in which such cell adhesion is undesirable or excessive, resulting in tissue damage instead of repair. For example, many pathologies (such as autoimmune and inflammatory diseases, shock and reperfusion injuries) involve abnormal adhesion of white blood cells. Such abnormal cell adhesion may also play a role in transplant and graft rejection. In addition, some circulating cancer cells appear to take advantage of the inflammatory mechanism to bind to activated endothelium. In such circumstances, modulation of selectin-mediated intercellular adhesion may be desirable.

Provided herein is a novel process for making Compound 16, an intermediate which is useful in the synthesis of E-selectin inhibitors.

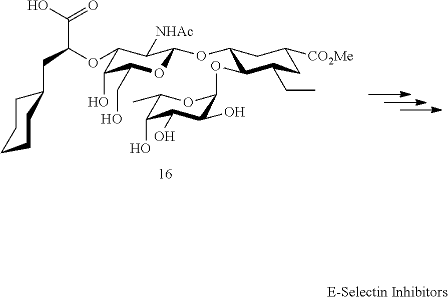

### BRIEF DESCRIPTION OF THE DRAWINGS

FIGS. 1a, 1b, and 1c illustrate the synthesis of Compound 16.

In some embodiments, a process for making Compound 16 is provided, wherein said process comprises hydrogenation of Compound 15.

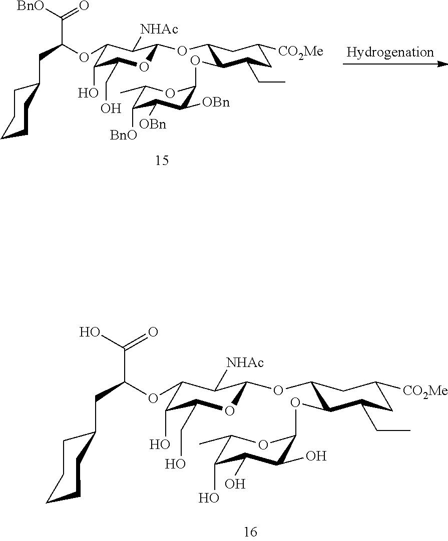

In some embodiments, the hydrogenation of Compound 15 comprises the use of H₂ and Pd/C. In some embodiments, the hydrogenation of Compound 15 is performed in the presence of at least one solvent. In some embodiments, the at least one solvent is chosen from alcohols. In some embodiments, the at least one solvent is 2-propanol. In some embodiments, the at least one solvent is chosen from esters and ethers. In some embodiments, the at least one solvent is THF. In some embodiments, the at least one solvent is water. In some embodiments, the hydrogenation of Compound 15 is performed in the presence of at least two solvents. In some embodiments, the at least two solvents are 2-propanol and THF. In some embodiments, the hydrogenation of Compound 15 is performed in the presence of at least three solvents. In some embodiments, the at least three solvents are 2-propanol, THE, and water.

In some embodiments, the process for making Compound 16 comprises MeO-trityl cleavage of Compound 14 to afford Compound 15.

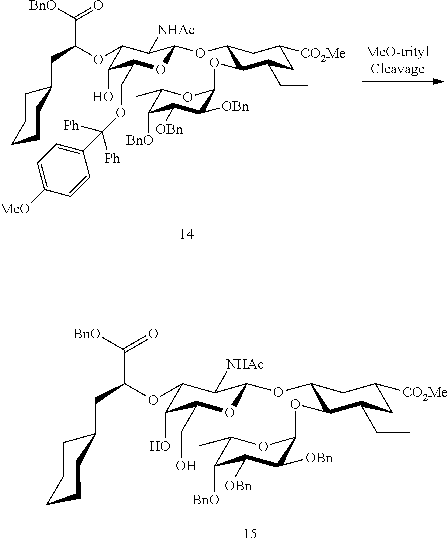

In some embodiments, the MeO-trityl cleavage of Compound 14 comprises the use of at least one acid. In some embodiments, the at least one acid is chosen from inorganic acids. In some embodiments, the at least one acid is chosen from organic acids. In some embodiments, the at least one acid is hydrochloric acid. In some embodiments, of the at least one acid is chosen from trifluoroacetic acid, trichloroacetic acid, formic acid, p-toluenesulfonic acid, and methanesulfonic acid. In some embodiments, the at least one acid is trichloroacetic acid.

In some embodiments, the MeO-trityl cleavage of Compound 14 is performed in the presence of at least one solvent. In some embodiments, the at least one solvent is chosen from alcohols. In some embodiments, the at least one solvent is methanol. In some embodiments, the at least one solvent is water. In some embodiments, the at least one solvent is dichloromethane. In some embodiments, the MeO-trityl cleavage of Compound 14 is performed in the presence of at least two solvent. In some embodiments, the at least two solvent are dichloromethane and methanol.

In some embodiments, the process for making Compound 16 comprises alloc cleavage and acylation of Compound 13 to afford Compound 14.

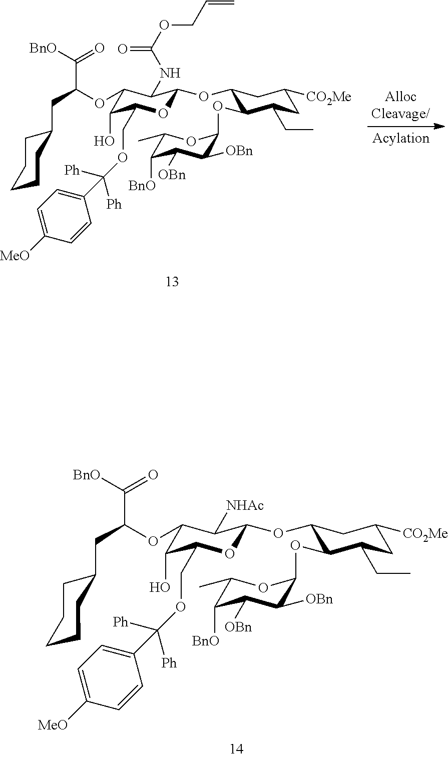

In some embodiments, the alloc cleavage/acylation of Compound 13 comprises the use of at least one base. In some embodiments, the at least one base is 4-methylmorpholine. In some embodiments, the alloc cleavage/acylation of Compound 13 comprises the use of at least one acid. In some embodiments, the at least one acid is acetic acid. In some embodiments, the alloc cleavage/acylation of Compound 13 comprises the use of at least one anhydride. In some embodiments, the at least one anhydride is acetic anhydride.

In some embodiments, the alloc cleavage/acylation of Compound 13 comprises the use of at least one phosphine. In some embodiments, the at least one phosphine is triphenylphosphine. In some embodiments, the alloc cleavage/acylation of Compound 13 comprises the use of at least one catalyst. In some embodiments, the at least one catalyst is Pd[(C₆H₅)₃P]₄.

In some embodiments, the alloc cleavage/acylation of Compound 13 is performed in the presence of at least one solvent. In some embodiments, the at least one solvent is dichloromethane. In some embodiments, the at least one solvent is toluene.

In some embodiments, the process for making Compound 16 comprises O-alkylation of Compound 11 with Compound 12 to afford Compound 13.

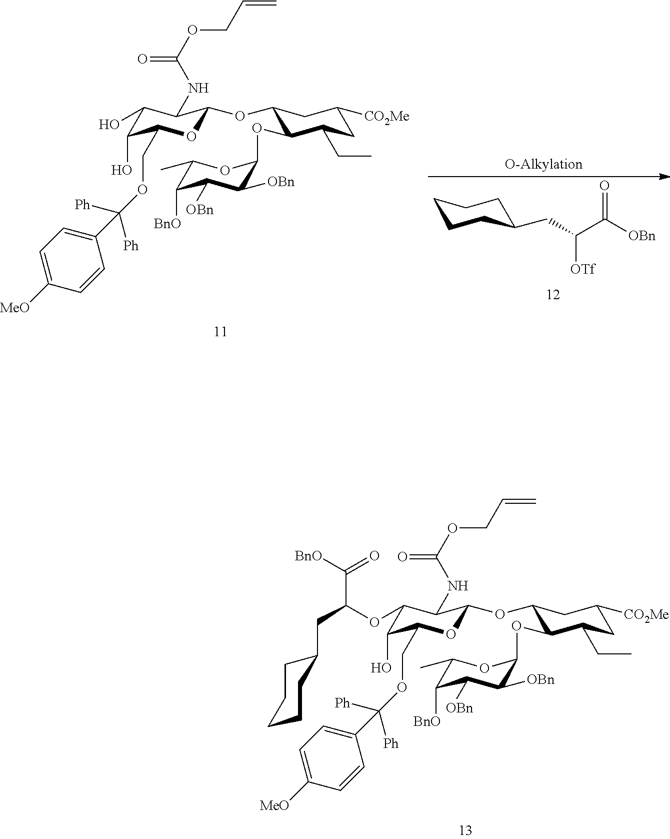

In some embodiments, the O-alkylation of Compound 11 comprises the use of at least one alkyltin. In some embodiments, the at least one alkyltin is dibutyltin(IV) oxide. In some embodiments, the O-alkylation of Compound 11 is performed in the presence of at least one solvent. In some embodiments, the at least one solvent is acetonitrile. In some embodiments, the at least one solvent is methanol. In some embodiments, the at least one solvent is toluene. In some embodiments, the O-alkylation of Compound 11 is performed in the presence of at least two solvents. In some embodiments, the at least two solvents are toluene and acetonitrile. In some embodiments, the O)-alkylation of Compound 11 comprises at least one fluoride. In some embodiments, the at least one fluoride is cesium fluoride.

In some embodiments, the process for making Compound 16 comprises methoxy-tritylation of Compound 10 to afford Compound 11.

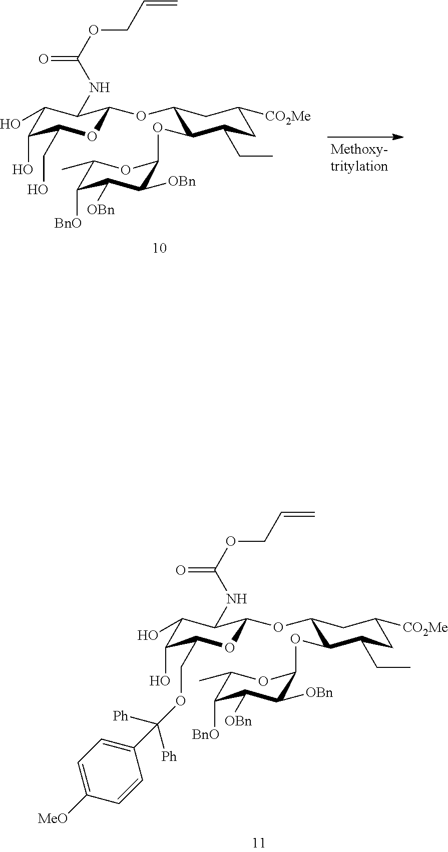

In some embodiments, the methoxy-tritylation of Compound 10 comprises the use of 4-MeO-trityl-Cl. In some embodiments, the methoxy-tritylation of Compound 10 comprises the use of at least one base. In some embodiments, the at least one base is chosen from DABCO, pyridine, and 2,6-lutidine. In some embodiments, the methoxy-tritylation of Compound 10 is performed in the presence of at least one solvent. In some embodiments, the at least one solvent is dichloromethane. In some embodiments, the at least one solvent is MeTHF. In some embodiments, the methoxy-tritylation of Compound 10 is performed in the presence of at least two solvents. In some embodiments, the at least two solvents are MeTHF and dichloromethane.

In some embodiments, the process for making Compound 16 comprises deacetylation of Compound 9 to afford Compound 10.

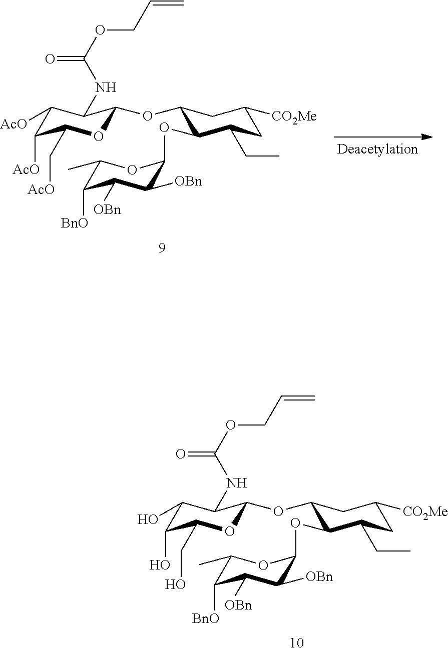

In some embodiments, the deacetylation of Compound 9 comprises the use of at least one base. In some embodiments, the at least one base is chosen from alkoxides. In some embodiments, the at least one base is NaOMe. In some embodiments, the deacetylation of Compound 9 is performed in the presenc of at least one solvent. In some embodiments, the at least one solvent is methanol. In some embodiments, the at least one solvent is methyl acetate. In some embodiments, the deacetylation of Compound 9 is performed in the presence of at least two solvents. In some embodiments, the at least two solvents are methanol and methyl acetate.

In some embodiments, Compound 10 is crystallized as an ethanol solvate. In some embodiments, Compound 10 is crystallized as an ethanol solvate in the presence of at least one solvent. In some embodiments, the at least one solvent is ethanol. In some embodiments, Compound 10 is crystallized as an ethanol solvate in the presence of at least two solvents. In some embodiments, the at least two solvents are ethanol and water. In some embodiments, crystalline Compound 10 is an ethanol solvate. In some embodiments, crystalline Compound 10 ethanol solvate is characterized by rod-like crystals.

In some embodiments, the process for making Compound 16 comprises glycosylation of Compound 6 with Compound 8 to afford Compound 9.

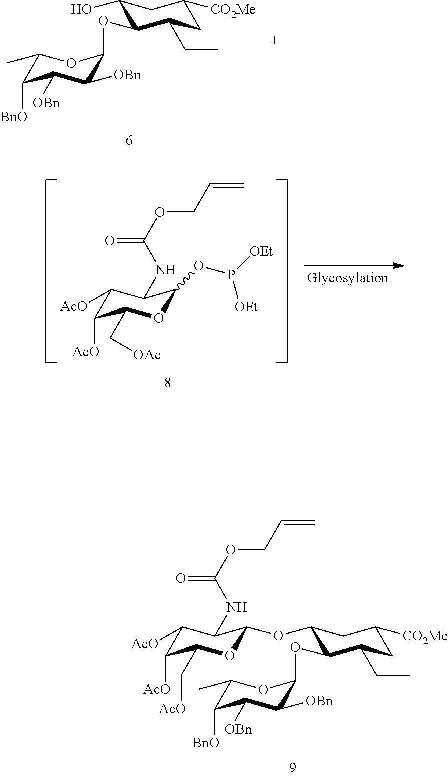

In some embodiments, the glycosylation of Compound 6 is performed in the presence of at least one solvent. In some embodiments, the at least one solvent is toluene. In some embodiments, the at least one solvent is dichloromethane. In some embodiments, the glycosylation of Compound 6 is performed in the presence of at least two solvents. In some embodiments, the at least two solvents are toluene and dichloromethane. In some embodiments, the glycosylation of Compound 6 comprises the use of at least one acid. In some embodiments, the at least one acid is triflic acid.

In some embodiments, the process for making Compound 8 comprises activation of Compound 7.

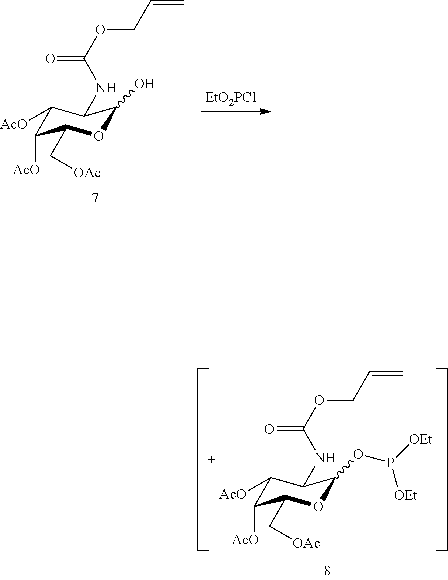

In some embodiments, the activation of Compound 7 comprises the use of at least one phosphite. In some embodiments, the at least one phosphite is chosen from chlorophosphites. In some embodiments, the at least one phosphite is diethylchlorophosphite. In some embodiments, the activation of Compound 7 is performed in the presence of at least one solvent. In some embodiments, the at least one solvent is toluene. In some embodiments, the activation of Compound 7 is performed in the presence of at least one organic base. In some embodiments, the at least one organic base is triethylamine.

In some embodiments, the process for making Compound 16 comprises TBDMS-deprotection of Compound 5 to afford Compound 6.

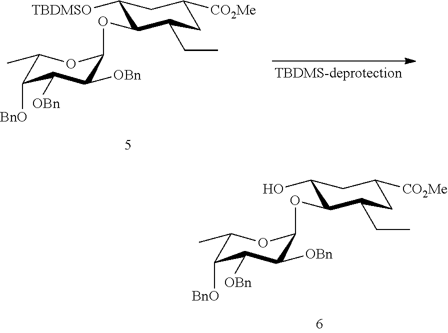

In some embodiments, the TBDMS-deprotection of Compound 5 comprises the use of at least one fluoride. In some embodiments, the at least one fluoride is TBAF. In some embodiments, the TBDMS-deprotection of Compound 5 is performed in the presence of at least one solvent. In some embodiments, the at least one solvent is THE. In some embodiments, the at least one solvent is ACN. In some embodiments, the TBDMS-deprotection of Compound 5 is performed in the presence of at least two solvents. In some embodiments, the at least two solvents are THF and ACN.

In some embodiments, Compound 6 is crystallized. In some embodiments, Compound 6 is crystallized in the presence of at least one solvent. In some embodiments, the at least one solvent is dichloromethane. In some embodiments, the at least one solvent is methanol. In some embodiments, the at least one solvent is water. In some embodiments, Compound 6 is crystallized in the presence of at least two solvents. In some embodiments, the at least two solvents are water and methanol.

In some embodiments, the process for making Compound 16 comprises fucosylation of Compound 3 with Compound 4b to afford Compound 5.

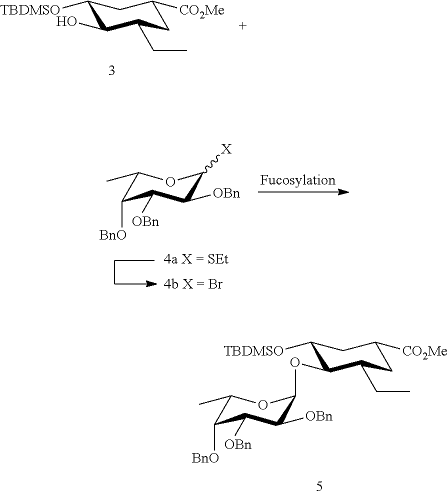

In some embodiments, the fucosylation of Compound 3 comprises the use of TBABr. In some embodiments, the fucosylation of Compound 3 comprises the use of at least one base. In some embodiments, the at least one base is DIPEA. In some embodiments, the fucosylation of Compound 3 is performed in the presence of at least one solvent. In some embodiments, the at least one solvent is MeTHF. In some embodiments, the at least one solvent is dichloromethane. In some embodiments, the fucosylation of Compound 3 is performed in the presence of at least two solvents. In some embodiments, the at least two solvents are MeTHF and dichloromethane.

In some embodiments, the process of making Compound 4b comprises reacting Compound 4a with Br₂. In some embodiments, the reaction of Compound 4a with Br₂ is performed in the presence of at least one solvent. In some embodiments, the at least one solvent is cyclohexane.

In some embodiments, the process for making Compound 16 comprises epoxide opening of Compound 2a to afford Compound 3.

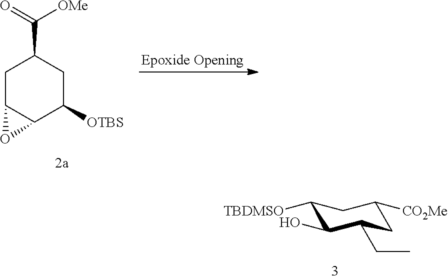

In some embodiments, the epoxide opening of Compound 2a comprises the use of at least one organogrignard reagent. In some embodiments, the at least one organogrignard reagent is chosen from ethyl magnesium halides. In some embodiments, the ethyl magnesium halide is ethyl magnesium bromide. In some embodiments, the ethyl magnesium halide is ethyl magnesium chloride. In some embodiments, 0.5 equivalents or more (relative to Compound 2a) of the ethyl magnesium chloride is used, such as 1 equivalent or more, 2 equivalents or more, 3 equivalents or more, 5 equivalents or more, 7 equivalents or more, 9 equivalents or more, 11 equivalents or more, 13 equivalents or more, and 15 equivalents or more, and may range from 0.5 equivalents to 25 equivalents, 3 equivalents to 20 equivalents, 5 equivalents to 20 equivalents, 5 equivalents to 15 equivalents, or 10 equivalents to 20 equivalents.

In some embodiments, the epoxide opening of Compound 2a comprises the use of at least one lewis acid. In some embodiments, the at least one lewis acid is chosen from boron trihalides and aluminum triflate. In some embodiments, the boron trihalide is chosen from boron trifluoride, boron trichloride, and boron tribromide. In some embodiments, the boron trihalide is boron trifluoride. In some embodiments, the boron trihalide is boron trichloride. In some embodiments, the boron trihalide is boron tribromide. In some embodiments, the boron trifluoride is boron trifluoride etherate. In some embodiments, the at least one lewis acid is aluminum triflate.

In some embodiments, 0.5 equivalents or more (relative to Compound 2a) of the lewis acid (e.g. boron trifluoride etherate) is used, such as 1 equivalent or more, 2 equivalents or more, 3 equivalents or more, 4 equivalents or more, 5 equivalents or more, 10 equivalents or more, and may range from 0.5 equivalents to 15 equivalents, 1 equivalent to 10 equivalents, 1 equivalent to 8 equivalents, 1 equivalent to 6 equivalents, 1 equivalent to 4 equivalents, 1 equivalent to 3 equivalents, 2 equivalents to 8 equivalents, 2 equivalents to 6 equivalents, 2 equivalents to 4 equivalents, 3 equivalents to 10 equivalents, 3 equivalents to 8 equivalents, or 3 equivalents to 6 equivalents.

In some embodiments, the epoxide opening of Compound 2a comprises the use of at least one copper(I) salt. In some embodiments, the at least one copper(I) salt is chosen from copper(I) halides, copper(I) triflates, copper(I) thiophenoxide, copper(I) cyanide, and 2-thienyl(cyano)copper lithium. In some embodiments, the copper(I) halide is copper(I) chloride. In some embodiments, the copper(I) halide is copper(I) bromide. In some embodiments, the copper(I) halide is copper(I) iodide. In some embodiments, the copper(I) bromide is copper(I) bromide-dimethyl sulfide complex. In some embodiments, the copper(I) triflate is copper(I) triflate benzene complex. In some embodiments, the copper(I) triflate is copper(I) triflate toluene complex. In some embodiments, the copper(I) cyanide is di(lithium chloride) complex.

In some embodiments, 0.01 equivalents or more (relative to Compound 2a) of the copper(I) salt (e.g. copper(I) bromide-dimethyl suflide complex) is used, such as 0.05 equivalent or more, 0.1 equivalent or more, 0.2 equivalent or more, 0.3 equivalent or more, 0.5 equivalent or more, 0.7 equivalent or more, 1 equivalent or more, 1.5 equivalent or more, 2 equivalent or more, 3 equivalent or more, 5 equivalent or more, 10 equivalent or more, and may range from 0.01 equivalents to 15 equivalents, 0.01 equivalents to 10 equivalents, 0.01 equivalents to 7 equivalents, 0.01 equivalents to 5 equivalents, 0.01 equivalents to 3 equivalents, 0.01 equivalents to 2 equivalents, 0.01 equivalents to 1 equivalents, 0.01 equivalents to 0.5 equivalents, 0.01 equivalents to 0.1 equivalents, 0.1 equivalents to 10 equivalents, 0.1 equivalents to 7 equivalents, 0.1 equivalents to 5 equivalents, 0.1 equivalents to 3 equivalents, 0.1 equivalents to 2 equivalents, 0.1 equivalents to 1 equivalent, 0.5 equivalents to 7 equivalents, 0.5 equivalents to 5 equivalents, 0.5 equivalents to 3 equivalents, 0.5 equivalents to 2 equivalents, 1 equivalent to 10 equivalents, 1 equivalent to 7 equivalents, 1 equivalent to 5 equivalents, or 1 equivalent to 3 equivalents

In some embodiments, the epoxide opening of Compound 2a comprises the use of at least one copper(I) salt, at least one ethyl magnesium balide, and at least one lewis acid In some embodiments, the at least one copper(I) salt is copper(I) bromide-dimethyl sulfide complex, the at least one ethyl magnesium halide is ethyl magnesium chloride, and the at least one lewis acid is boron trifluoride etherate. In some embodiments, about 0.01 equivalents (relative to Compound 2a) of the copper(I) bromide-dimethyl suflide complex, about 9 equivalents (relative to Compound 2a) of the ethyl magnesium bromide, and about 3 equivalents (relative to Compound 2a) of the boron trifluoride etherate complex is used. In some embodiments, about 3 equivalents (relative to Compound 2a) of the copper(I) bromide-dimethyl suflide complex, about 9 equivalents (relative to Compound 2a) of the ethyl magnesium bromide, and about 3 equivalents (relative to Compound 2a) of the boron trifluoride etherate complex is used. In some embodiments, about 5 equivalents (relative to Compound 2a) of the copper(I) bromide-dimethyl suflide complex, about 15 equivalents (relative to Compound 2a) of the ethyl magnesium bromide, and about 5 equivalents (relative to Compound 2a) of the boron trifluoride etherate complex is used.

In some embodiments, the epoxide opening of Compound 2a comprises the use of copper(I) bromide-dimethyl sulfide complex and ethyl magnesium chloride where the molar ratio of the copper(I) bromide-dimethyl sulfide complex to the ethyl magnesium chloride is about 1 to 3. In some embodiments, the molar ratio of the copper(I) bromide-dimethyl sulfide complex to the ethyl magnesium chloride is about 1 to 2. In some embodiments, the molar ratio of the copper(I) bromide-dimethyl sulfide complex to the ethyl magnesium chloride is about 1 to 1.5. In some embodiments, the molar ratio of the copper(I) bromide-dimethyl sulfide complex to the ethyl magnesium chloride is about 1 to 1. In some embodiments, the molar ratio of the copper(I) bromide-dimethyl sulfide complex to the ethyl magnesium chloride is about 1 to 4. In some embodiments, the molar ratio of the copper(I) bromide-dimethyl sulfide complex to the ethyl magnesium chloride is about 1 to 5.

In some embodiments, the epoxide opening of Compound 2a is performed in the presence of at least one solvent. In some embodiments, the at least one solvent is a polar aprotic solvent. In some embodiments, the at least one solvent is THF. In some embodiments, the at least one solvent is cyclopentylmethyl ether.

In some embodiments, the epoxide opening of Compound 2a is performed at a temperature within the range of −100° C. to 30° C., such as −100° C. to 10° C., −100° C. to 0° C., −100° C. to −20° C., −100° C. to −40° C., −100° C. to −60° C., −20° C. to 30° C., −20° C. to 20° C., −20° C. to 10° C. −20° C. to 0° C., −10° C. to 25° C., −10° C. to 15° C., −10° C. to 5° C. In some embodiments, the epoxide opening of Compound 2a is performed at about −78° C.

In some embodiments, the epoxide opening of Compound 2a is performed in the presence of Compound 2b. In some embodiments, the molar ratio of Compound 2a to 2b prior to epoxide opening is greater than 7 to 1, such as greater than 7.5 to 1, greater than 8 to 1, greater than 9 to 1, greater than 10 to 1, greater than 11 to 1, greater than 15 to 1, greater than 25 to 1, or greater than 50 to 1.

In some embodiments, the epoxide opening of Compound 2a comprises the use of Compound 2a prepared by oxidation of Compound 1 without chromatography.

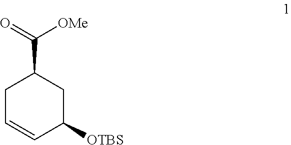 [SMILES: COC(=O)[C@@H]1CC=C[C@H](O[Si](C)(C)C(C)(C)C)C1]

In some embodiments, the process for making Compound 16 comprises epoxidation of Compound 1 to afford Compound 2a.

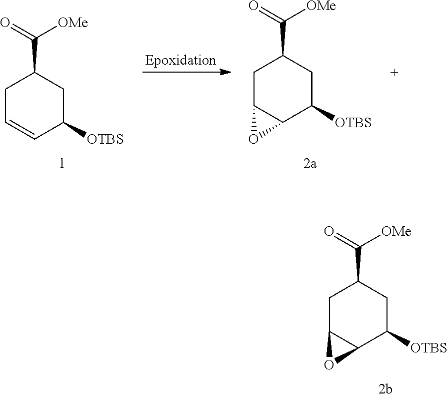 [SMILES: COC(=O)[C@@H]1CC=C[C@H](O[Si](C)(C)C(C)(C)C)C1 ; COC(=O)[C@@H]1C[C@H]2O[C@H]2[C@H](O[Si](C)(C)C(C)(C)C)C1 ; COC(=O)[C@@H]1C[C@@H]2O[C@@H]2[C@H](O[Si](C)(C)C(C)(C)C)C1]

In some embodiments, the epoxidation of Compound 1 (WO2013/096926) comprises the use of potassium peroxymonosulfate (e.g. Oxone). In some embodiments, 0.5 equivalents or more (relative to Compound 1) of potassium peroxymonosulfate is used, such as 1 equivalent or more, 2 equivalents or more, 3 equivalents or more, 4 equivalents or more, 5 equivalents or more, and may range from 0.5 equivalents to 10 equivalents, 1 equivalent to 8 equivalents, 1 equivalent to 6 equivalents, 1.5 equivalents to S equivalents, or 2 equivalents to 4 equivalents.

In some embodiments, the epoxidation of Compound 1 comprises the use of at least one base. In some embodiments, the at least one base is chosen from metal carbonates. In some embodiments, the metal carbonate is NaHCO₃. In some embodiments, 1 equivalent or more (relative to Compound 1) of NaHCO₃ is used, such as 2 equivalent or more, 3 equivalents or more, 4 equivalents or more, 5 equivalents or more, 8 equivalents or more, 10 equivalents or more and may range from 1 equivalent to 20 equivalents, 1 equivalent to 10 equivalents, 1 equivalent to 7 equivalents, 1 equivalent to 5 equivalents, 3 equivalents to 15 equivalents, 3 equivalents to 9 equivalents, 3 equivalents to 6 equivalents, 4 equivalents to 12 equivalents, 4 equivalents to 8 equivalents, or 4 equivalents to 6 equivalents.

In some embodiments, the epoxidation of Compound 1 comprises the use of potassium peroxymonosulfate (e.g. Oxone) and the use of at least one base. In some embodiments, the epoxidation of Compound 1 comprises the use of potassium peroxymonosulfate (e.g. Oxone) and NaHCO₃. In some embodiments, about 2.5 equivalents (relative to Compound 1) of the potassium peroxymonosulfate (e.g. Oxone) and about 4.5 equivalents (relative to Compound 1) of the NaHCO₃ is used.

In some embodiments, the epoxidation of Compound 1 is performed in the presence of at least one solvent. In some embodiments, the at least one solvent is acetone. In some embodiments, the at least one solvent is water. In some embodiments, the epoxidation of Compound 1 is performed in the presence of at least two solvents. In some embodiments, the at least two solvents are acetone and water.

In some embodiments, the epoxidation of Compound 1 is performed at a temperature within the ranges of −10° C. to 50° C., such as −10° C. to 40° C. −10° C. to 30° C., −10° C. to 20° C., −10° C. to 10° C., −5° C. to 45° C., −5° C. to 35° C., −5° C. to 25° C., −5° C. to 15° C., −5° C. to 10° C., −5° C. to 5° C. In some embodiments, the epoxidation of Compound 1 is performed at about 0° C.

In some embodiments, the epoxidation of Compound 1 results in the formation of Compound 2a and 2b. In some embodiments, the molar ratio of Compound 2a to 2b formed as a result of epoxidation is greater than 7 to 1, such as greater than 7.5 to 1, greater than 8 to 1, greater than 9 to 1, greater than 10 to 1, greater than 11 to 1, greater than 15 to 1, greater than 25 to 1, or greater than 50 to 1.

In some embodiments, the process for making Compound 16 comprises at least one of the following steps:
(a) hydrogenation of Compound 15;
(b) MeO-trityl cleavage of Compound 14;
(c) alloc cleavage/acylation of Compound 13;
(d) O-alkylation of Compound 11;
(e) methoxy-tritylation of Compound 10;
(f) deacetylation of Compound 9;
(g) glycosylation of Compound 6;
(h) TBDMS-deprotection of Compound 5; and
(i) fucosylation of Compound 3.
(j) epoxide opening of Compound 2a.
(k) epoxidation of Compound 1.

In some embodiments, step d above comprises the O-alkylation of Compound 11 with Compound 12 to form Compound 13. In some embodiments, step g above comprises the glycosylation of Compound 6 with Compound 8 to form Compound 9.

In some embodiments, the process for making Compound 16 comprises at least two steps chosen from steps (a)-(k) above. In some embodiments, the process for making Compound 16 comprises at least three steps chosen from steps (a)-(k) above. In some embodiments, the process for making Compound 16 comprises at least four steps chosen from steps (a)-(k) above. In some embodiments, the process for making Compound 16 comprises at least five steps chosen from steps (a)-(k) above. In some embodiments, the process for making Compound 16 comprises at least six steps chosen from steps (a)-(k) above. In some embodiments, the process for making Compound 16 comprises at least seven steps chosen from steps (a)-(k) above. In some embodiments, the process for making Compound 16 comprises at least eight steps chosen from steps (a)-(k) above. In some embodiments, the process for making Compound 16 comprises at least nine steps chosen from steps (a)-(k) above. In some embodiments, the process for making Compound 16 comprises at least ten steps chosen from steps (a)-(k) above. In some embodiments, the process for making Compound 16 comprises each of steps (a)-(k) above.

In some embodiments, the process for making Compound 16 comprises at least step (j) above. In some embodiments, the process for making Compound 16 comprises at least step (k) above. In some embodiments, the process for making Compound 16 comprises at least steps (j) and (k) above.

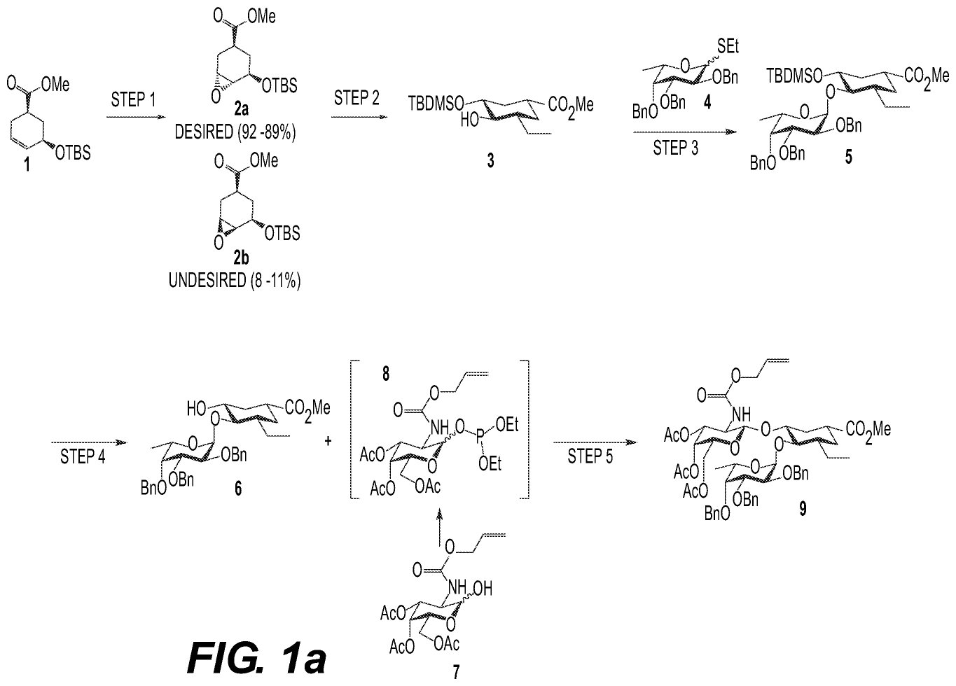

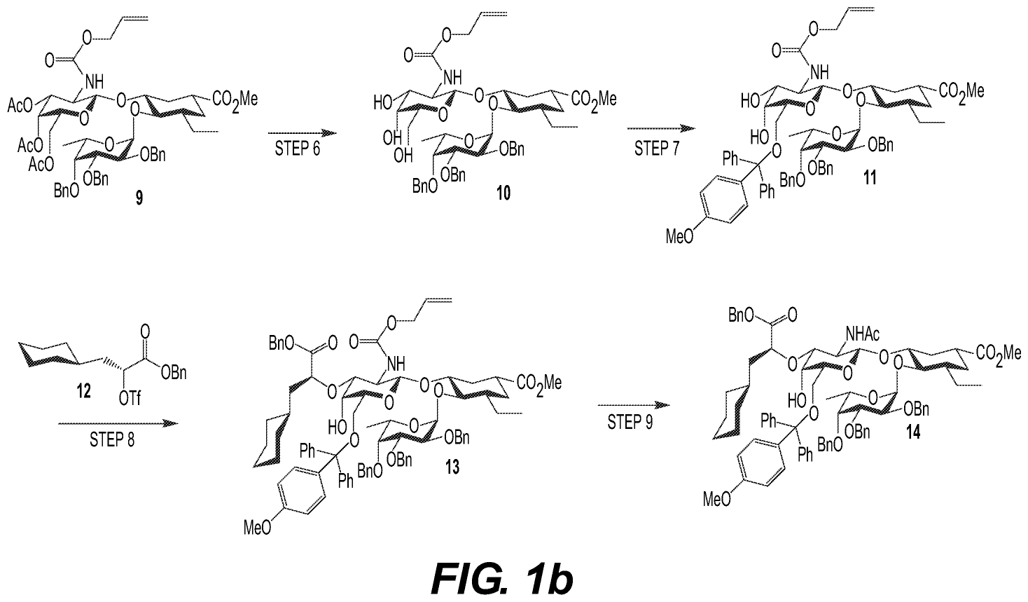

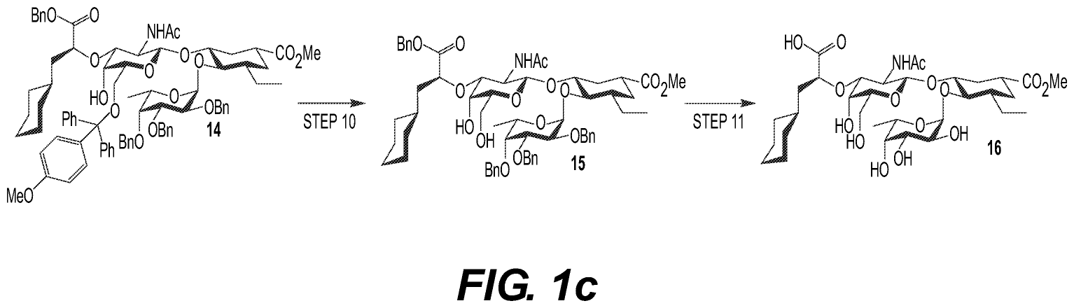

Compound 16 may be prepared according to the General Reaction Scheme shown in FIGS. 1a, 1b, and 1c. It is understood that one of ordinary skill in the art may be able to make these compounds by similar methods or by combining other methods known to one of ordinary skill in the art. In general, starting components may be obtained from sources such as Sigma Aldrich, Lancaster Synthesis, Inc., Maybridge, Matrix Scientific, TCI, and Fluorochem USA, etc and/or synthesized according to sources known to those of ordinary skill in the art (see, for example, Advanced Organic Chemistry: Reactions, Mechanisms, and Structure, 5th edition (Wiley, December 2000)) and/or prepared as described herein.

Analogous reactants to those described herein may be identified through the indices of known chemicals prepared by the Chemical Abstract Service of the American Chemical Society, which are available in most public and university libraries, as well as through on-line databases (the American Chemical Society, Washington, D.C., may be contacted for more details) Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes: “Organic Reactions” (1942-2000) John Wiley & Sons, in over 55 volumes; and “Chemistry of Functional Groups” John Wiley & Sons, in 73 volumes.

### EXAMPLE

### Synthesis Of Compound 16

### Step 1

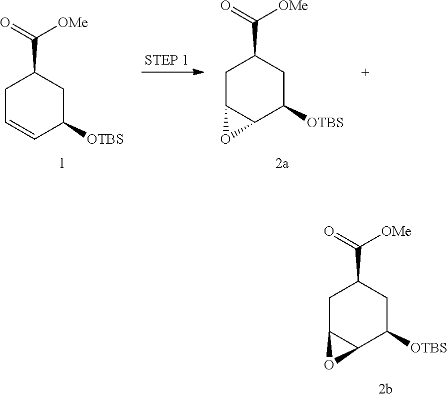 [SMILES: COC(=O)[C@@H]1CC=C[C@H](O[Si](C)(C)C(C)(C)C)C1 ; COC(=O)[C@@H]1C[C@H]2O[C@H]2[C@H](O[Si](C)(C)C(C)(C)C)C1 ; COC(=O)[C@@H]1C[C@@H]2O[C@@H]2[C@H](O[Si](C)(C)C(C)(C)C)C1]

Compound 2a/2b: Olefin 1 (described in Magnani et al WO 2013/096926, 8.1 g, 0.03 mol) was dissolved in acetone (90 mL) at room temperature. Solid NaHCO; (12.0 g, 0.14 mol) was added. The mixture was cooled to 0° C. Oxone (24 g, 0.08 mmol) dissolved in 150 mL of water was added dropwise (1 mL/min). The reaction mixture was vigorously stirred at 0° C. until TLC indicated consumption of olefin 1. The reaction mixture was transferred to a separatory funnel and extracted 3 times with MTBE. The combined organic phases were extracted 2 times with water. The organic phase was dried over MgSO₄, filtered and concentrated to afford mixture of epoxides 2a/2b (8.0 g, 93% yield). LCMS analysis indicated around 11% of undesired epoxide 2b. LC-MS m/z=287.2 (M+1).

### Step 2

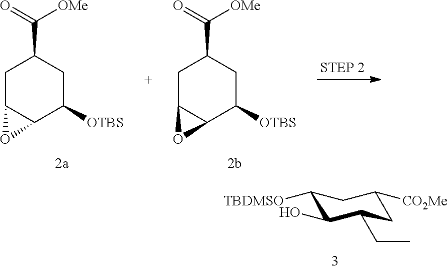

Compound 3: To a stirred suspension of CuBrDMS (10.8 g, 0.052 mol, 5.0 equiv.) in anhydrous THF (240 mL) cooled to −78° C. was added dropwise (over a period of 20 minutes) EtMgCl (Acros Organics 2.7 M in THF, 58.2 mL, 0.157 mol 15.0 equiv.) The mixture was stirred for 90 min at −78° C. to form the cuprate. Epoxide 2a (3.0 g, 0.01 mmol, 1.0 equiv.) dissolved in anhydrous THF (30 mL) was added, followed by dropwise addition of BF₃·Et₂O (6.46 mL, 0.052 mol, 5.0 equiv.) while the mixture was maintained at −78° C. The reaction mixture was stirred at this temperature for 30 min. The reaction was quenched at −78° C. by dropwise addition of a mixture of methanol (30 mL) and triethylamine (12 mL) and allowed to warm to room temperature. The reaction mixture was diluted with sat'd aq. NH₄Cl solution (200 mL) and NH₄OH (30 mL) and stirred vigorously for 10 min. The layers were separated, and the organic layer was extracted with MTBE (2×), washed with brine, dried (Na₂SO₄), filtered through celite and concentrated in vacuo to obtain the desired product 3 (3.03 g, 92% yield). Alcohol 3 was used for the next step without further purification. LC-MS m/z=317.2 (M+1).

Compound 3 (using catalytic CuBr·DMS): To a stirred suspension of CuBr·DMS (576 mg, 2.8 mmol, 0.1 equiv.) in anhydrous THF (200 mL) cooled to −78° C. was added dropwise (over a period of 20-30 minutes) EtMgCl (Acros Organics, 2.7 M in THF, 93 mL, 252 mmol 9.0 equiv.). The mixture was stirred for 60 min at −78° C. to form the cuprate. Crude epoxides 2a/2b (8.0 g, 28 mmol, 1.0 equiv.) dissolved in anhydrous THF (60 mL) were added, followed by dropwise addition of BF₃·Et₂O (10.4 mL, 84 mmol, 3.0 equiv.) at −78° C. The reaction mixture was stirred at this temperature for 45 min. The reaction was quenched at −78° C. by dropwise addition of a mixture of methanol (80.0 mL) and triethylamine (35 mL) then allowed to warm to room temperature. The reaction mixture was diluted with sat'd aq. NH₄Cl solution (300 mL) and NH₄OH (80 mL) and stirred vigorously for 10 min. The layers were separated, and the organic layer was extracted with MTBE (2×), washed with brine, dried (Na₂SO₄), filtered through celite and concentrated in vacuo to obtain crude 3 (7.9 g, 89% yield).

Compound 3 (using cyclopentylmethyl ether as solvent): To a stirred suspension of CuBr·DMS (216 mg, 1.05 mmol, 3.0 equiv.) in anhydrous CPME (Cyclopentylmethyl ether 5 mL) cooled to −78° C. was added dropwise (over a period of 5 minutes) EtMgCl (2.7 M in THF, 1.17 mL, 3.15 mmol 9.0 equiv.). The mixture was stirred for 60 min at −78° C. to prepare the cuprate. The epoxide 2a (100 mg, 0.35 mmol, 1.0 equiv.) dissolved in anhydrous CPME (1.0 mL) was added, followed by dropwise addition of BF₃Et₂O) (0.13 mL, 1.05 mmol, 3.0 equiv.) at −78° C. The mixture was stirred at −78° C. for 30 min. The reaction was quenched at −78° C. by dropwise addition of a mixture of methanol (1.0 mL) and triethylamine (0.4 mL) and allowed to warm to room temperature. The reaction mixture was diluted with sat'd aq. NH₄Cl solution (7 mL) and NH₄OH (1 mL) and stirred vigorously for 10 min. The layers were separated, and the organic layer was extracted with MTBE (2×), washed with brine, dried (Na₂SO₄), filtered through celite and concentrated in vacuo to obtain compound 3 (106 mg, 96%, Purity: 100%)

### Step 3

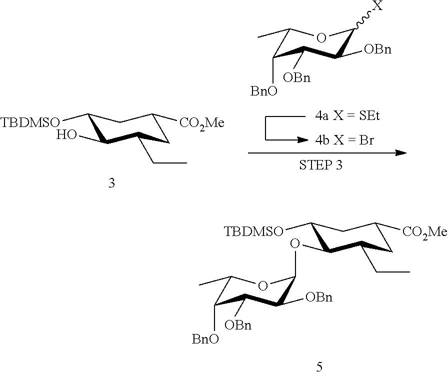

Compound 5: 5.7 g of Compound 4a (1.20 eq) were dissolved in anhydrous cyclohexane (50 mL) and concentrated. Anhydrous cylcohexane was added (50 mL) and stripped off again and the residue was dried under vacuum for 30 min. To a cooled (0° C.) solution of dried thioglycoside in anhydrous CH₂Cl₂ (13 mL) was added a solution of bromine (0.59 mL, 0.01 mol, 1.15 equiv.) in anhydrous CH₂Cl₂ (1.5 mL) over 10 min and the mixture was stirred for 60 min at 0° C. before cyclohexene (2.9 mL, 0.03 mol, 3.0 equiv.) was added over 5 min. The mixture was stirred for another 30 min at 0° C. to give the donor solution.

Alcohol 3 (3.03 g, 0.01 mol, 1.0 equiv.) and TBABr (3.09 g, 0.01 mol, 1.0 equiv) were dissolved in anhydrous cyclohexane (20 mL) then concentrated. Anhydrous cylcohexane (20 mL) was added and distilled off again and the mixture was dried under vacuum for 1 h. To freshly activated molecular sieves (powdered, 4A, 9.0 g) was added 3.0 mL anhydrous CH₂Cl₂, followed by the dried mixture of alcohol 3 and TBABr as a solution in anhydrous CH₂Cl₂ (9 mL) and N,N-Diisopropylethylamine (5.0 mL, 0.03 mol, 3.0 equiv). The suspension was allowed to stir at rt for 2 h to give the acceptor solution.

The acceptor solution was added to the donor solution at 0° C. and the reaction was allowed to stir at rt for 2 days after which the mixture was filtered through Celite to remove the molecular sieves. The filtrate was washed successively with water, 15% aq. citiric acid, 10% aq. NaHCO₃ and finally with water again, dried over Na₂SO₄ and concentrated to give crude 5 as a yellow oil which was used for the next step without further purification.

Step 4

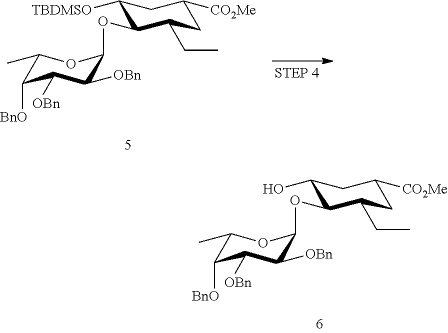

Compound 6: To crude 5 was added a solution of TBAF (1.0 M in THF, 30 mL, 0.03 mol, 3.0 equiv.) and the mixture was heated at 55° C. for 18 h after which it was concentrated in vacuo. The residue was dissolved in CH₂Cl₂ (50 mL), transferred to a separatory funnel, and washed with water (50 mL). The phases were separated and the aqueous phase was extracted with CH₂Cl₂ (2×30 mL.). The combined organic extracts were dried over Na₂SO₄, filtered and concentrated. The residue was crystallized from methanol and water to give product 6 as an off-white solid. Since recrystallization was incomplete, the mother liquor was subjected to flash chromatography to isolate remaining product 6 (4.4 g total, 71% yield).

¹H NMR (400 MHZ, Chloroform-d) δ 7.47-7.24 (m, 15H), 5.05-4.97 (m, 2H), 4.89-4.61 (m, 6H), 4.19-4.09 (m, 2H), 3.98 (dd, J=10.2, 2.7 Hz, 1H), 3.75-3.66 (m, 4H), 3.51 (s, 1H), 3.00 (dd, J=10.3, 8.4 Hz, 1H), 2.37 (tt, J=12.6, 3.3 Hz, 1H), 2.25 (ddt, J=13.0, 5.3, 3.0 Hz, 1H), 2.06 (dp, J=14.3, 3.5 Hz, 2H), 1.47 (dt, J=24.1, 12.2 Hz, 2H), 1.23-1.14 (m, 4H), 0.81 (t, J=7.4 Hz, 3H). LC-MS m/z=619.2 (M+1), 641.2 (M+Na).

Step 5

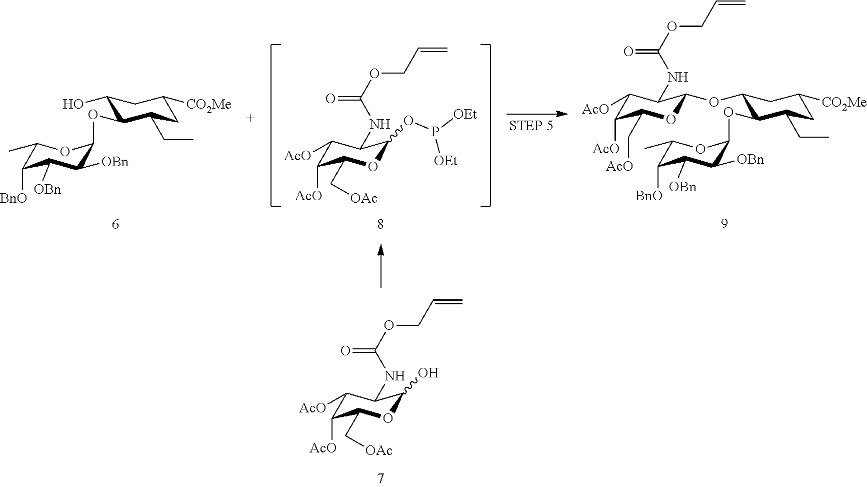

Compound 8: To Compound 7 (1.50 eq corr., 45.32 g n.corr./42.47 g corr.) toluene (8 vol) was added, then 5 vol of solvent were distilled off at Ta=55° C./130-60 mbar. Toluene (2 vol) was added and 2 vol of solvent were distilled off at Ta=55° C. The concentrate was diluted with toluene (5.5 vol). After cooling to Ti=0-5° C. triethylamine (2.05 eq) was added. Diethyl chlorophosphite (0.93 eq) was added at Ti=0-3° C. over 30 min to the reaction mixture (exotherm). The mixture was stirred at Ti=0° C. for 30 min. A second portion of diethyl chlorophosphite (0.13 eq) was added at Ti=0-5° C. over 10 min. The mixture was stirred at Ti=0° C. for 30 min. A third portion of diethyl chlorophosphite (0.09 eq) was added at Ti=0-5° C. over 7 min. The mixture was stirred at Ti=0° C. for 30 min.

The reaction mixture was filtered off from the solids (TEAxHCl) at Ti=1° C. under nitrogen atmosphere and washed with cold toluene (3 vol). Filtrate was fine filtered over 0.2 μm tip filter. Filtrate was fine filtered a second time over 0.2 μm tip filter. The filtrate was stored overnight at Ta=4° C. and subsequently filtered a third time over 0.2 μm tip filter. The phosphite solution was stored in the freezer for the following glycosylation experiment.

Compound 9: 126.41 g glycosylphosphite solution (33.1 mmol Compound 8, 1.28 eq.) was placed in a 500 mL flask and charged with 16.03 g Compound 6 (15.95 g, 25.78 mmol) and 32 mL (2 vol) of toluene. The solution was concentrated on the rotavap at Tj=50° C./100-4 mbar removing 175 mL (˜11 vol) of toluene. The resulting solid residue was dissolved in 96 mL. (6 vol) DCM and transferred into a 3 necked-flask.

The reaction was initiated by dosing 3.53 g (23.5 mmol, 0.91 eq.) of trifluoromethanesulfonic acid over 30 min at Ti=−30° C. The reaction was quenched after 7.5 h charging 4.756 g (46.94 mmol, 1.82 eq.) of NEt₃. The reaction mixture (184.16 g clear orange solution) was stored at T=−20° C. until further processing.

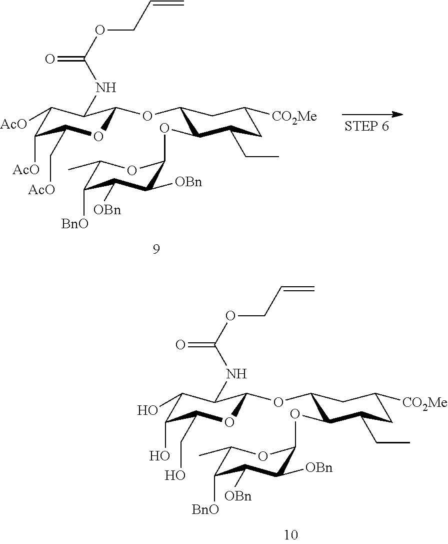

Compound 10: The crude Compound 9 reaction mixture was concentrated by distilling 5 vol off at Ta=55° C./600-100 mbar. Toluene (4 vol) was added, followed by a mixture of 23.1% NaCl-soln. (2.5 vol) and 7.4% NaHCO₃-soln. (2.5 vol). Phases were separated and the aqueous layer (AP 1 #1, pH 9) was re-extracted with toluene (5 vol). The volume of the combined organic layers (OP 1) was determined to 198 mL. OP 1 was concentrated to 4.3 vol concentrate volume at Ta=58° C./200-79 mbar by distilling 132 mL of solvent off. The concentrate was diluted with methanol (3.5 vol) and methyl acetate (1 vol) was added. NaOMe 30% in MeOH (0.60 eq) was added and the addition tank was rinsed with methanol (0.5 vol). The reaction mixture was stirred 3 h at Ti=20° C.

The reaction mixture was quenched by the addition of acetic acid (0.60 eq) over 5 min at Ti=20° C. to reach a pH of 5-6. 5 vol of solvent were distilled off at Ta=56° C./300-260 mbar. Ethyl acetate (2.5 vol) was added and 2.5 vol were distilled off at Ta 58° C./200 mbar. Ethyl acetate (5 vol), 23.1% NaCl-soln. (2.5 vol) and water (2.5 vol) were added and after stirring phases were separated (->AP 2 #1 pH 6, OP 2 #1). The aqueous layer (AP 2 #1) was re-extracted with ethyl acetate (3 vol) (->OP 2 #2). The combined organic layers were washed with 23.1% NaCl-soln. (5 vol) and the volume of the organic layer (OP 3 #1) was determined to 180 mL.

OP 3 #1 was concentrated to 4.0 vol concentrate volume at Ta=60° C./330-300 mbar by distilling 116 mL of solvent off. 2-Methyl-2-butanol (5 vol) was added at Tj=60° C. (still a solution). 2.75 vol of solvent were distilled off at Tj=67° C./280-195 mbar resulting in a slightly turbid solution.

The solution was warmed to Ti=70° C. over 30 min. The solution was then allowed to cool to room temperature over 100 min. Precipitation has started at Ti approx. 33° C. The suspension was stirred at Ti=20° C. for 85 min. Then n-Heptane (8 vol) was added at Ti=20° C. over 50 min and the suspension was cooled to Ti=10° C. over 25 min and stirred 3 h at this temperature. Filtration of suspension (2 min), washing of filter cake with a mixture of 2-Methyl-2-butanol/n-Heptane (0.7 vol/1.4 vol at 10° C.) and finally with n-Heptane (3 vol) cooled to Ti=10° C. Drying of the product on nutsch filter in vacuum/nitrogen overnight and further on rotavap at Ta=45° C. for 6 h to a dry weight content of 97.22%. 17.00 g n.corr./16.527 g LOD corr. (Y: 73.91%).

¹H NMR (Chloroform-d) δ 7.23-7.43 (m, 17H), 5.90 (ddt, J=17.2, 10.4, 5.8 Hz, 1H), 5.31 (dq, J=17.1, 1.5 Hz, 1H), 5.24 (dd, J=10.4, 1.3 Hz, 1H), 5.10 (d, J=3.3 Hz, 1H), 4.59-5.01 (m, 9H), 4.53-4.58 (m, 2H), 4.44 (d, J=7.9 Hz, 1H), 4.00-4.12 (m, 2H), 3.83-3.94 (m, 2H), 3.71-3.82 (m, 4H), 3.68 (s, 3H), 3.32-3.35 (m, 1H), 2.34 (tt, J=12.2, 3.2 Hz, 1H), 2.20 (d, J=13.2 Hz, IH), 1.91-2.05 (m, 2H), 1.40-1.60 (m, 3H), 1.16-1.30 (m, 4H), 1.12 (d, J=6.6 Hz, 4H), 0.92 (t, J=7.6 Hz, 1H), 0.81 (t, J=7.4 Hz, 3H). MS: Calculated for C₄₇H₆₁NO₁₄=863.99; Found m/z=886.4 (M+Na⁺).

### Step 7

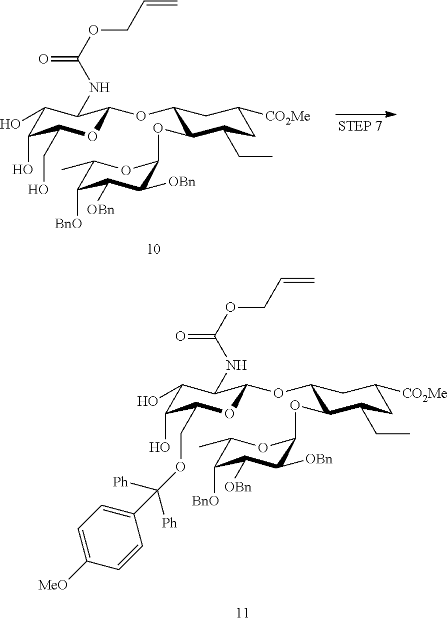

Compound 11: Compound 10 (25.00 g) was dissolved in DCM (6 vol). Solvent (4 vol) was distilled off at Tj=50° C./vac. DCM (6 vol) was added and the same volume of solvent was distilled off. DCM (6 vol) was added and the same volume of solvent was distilled off. The clear yellowish concentrate was diluted with DCM (4 vol) and cooled to ambient temperature under nitrogen. 2,6-Lutidine (1.8 eq) was added. 4-MeO-trityl chloride (1.03 eq) was added and in three portions and rinsed with DCM (0.S vol) into the reaction mixture and stirred at ambient temperature for 1 h.

Water (3 vol) was charged followed by Me-THF (6 vol) and 6 vol of solvent were distilled off. Me-THF (6 vol) was added and the same amount of solvent was distilled off. Citric acid 15% w/w (3 vol) was added and the mixture vigorously stirred. The phases were separated and the organic phase was washed with a mixture of water (3 vol), brine (3 vol) and sat. NaHCO₃ aq. (1 vol). The phases were separated and the pH of the aqueous phase was measured to be 7. The organic phase was washed with half concentrated aqueous NaCl (6 vol) to yield 140 mL of organic phase.

The product solution was concentrated to 4 vol by distillative removal of approx. 50 mL of solvent at Tj=45° C./250 mbar. The concentrate was warmed to Ti=40° C. and n-heptane (12 vol) was added over 30 min at the same temperature. The resulting suspension was heated to Ti=60° C. to dissolve crusts from the wall of the flask and held at this temperature for 25 min. The suspension was cooled to 20° C. over 2 h and stirred at this temperature overnight. The solid was filtered over a 250 mL turn over fritt P3. The filter cake was rinsed with mother liquor and n-heptane (2.3 vol) and dried in vacuum under nitrogen flow for 5 h and further on the rotavap at Tj=33° C. overnight. 30.03 g n.corr./29.89 g LOD corr. (Y 93.8% corr.).

¹H NMR (Chloroform-d) δ 1H NMR (CHLOROFORM-d) Shift: 7.09-7.47 (m, 28H), 6.76-6.82 (m, 2H), 5.83-5.99 (m, 1H), 5.32 (dd, J=17.2, 1.5 Hz, 1H), 5.24 (dd, J=10.3, 1.4 Hz, 1H), 4.77-5.00 (m, 4H), 4.44-4.75 (m, 7H), 4.10-4.21 (m, 2H), 3.98-4.09 (m, 2H), 3.75-3.95 (m, 4H), 3.61-3.70 (m, 6H), 3.54-3.60 (m, 1H), 3.37-3.50 (m, 2H), 3.27-3.37 (m, 2H), 2.15-2.37 (m, 2H), 1.93-2.14 (m, 2H), 1.36-1.56 (m, 2H), 1.05-1.29 (m, 5H), 0.73-0.86 (m, 3H). MS: Calculated for C₆₇H₇₇NO₁₅=1136.33, Found m/z=1158.5 (M+Na⁺).

Step 8

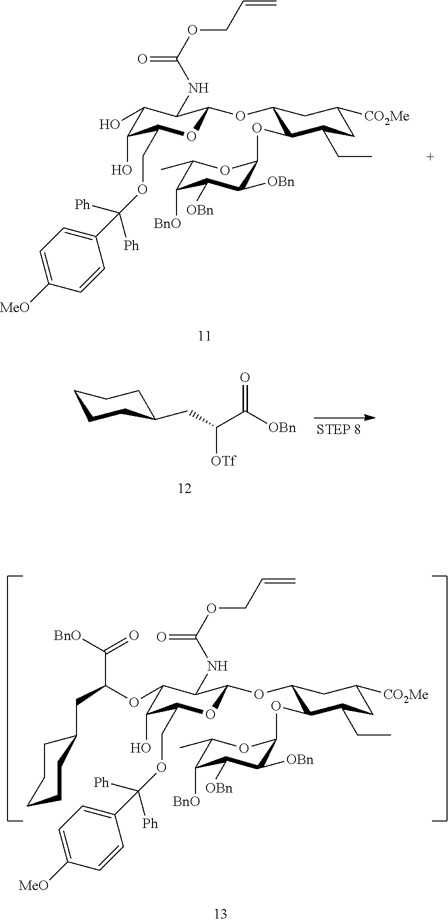

Compound 13: Compound 11 (20.45 g, 1 wt.), dibutyltin(IV) oxide (0.37 wt./1.7 eq), methanol (4 vol) and toluene (2 vol) were heated to reflux at Tj=82° C. and stirred under reflux for 2 h. Solvent (3 vol) was removed via distillation at Tj=65° C./320 mbar). Toluene (3 vol) was added and the solution was stirred under reflux at Tj=82° C. for 75 min. Solvent (4 vol) was removed by distillation at Tj=65° C./400-140 mbar. Toluene (3 vol) was added and solvent (3 vol) was removed via distillation at Tj=65° C./130 mbar). Toluene (3 vol) was added and solvent (3 vol) was removed via distillation at Tj=65° C./105 mbar).

Acetonitrile (5 vol) was added to the concentrate at Ti=20° C. Compound 12 in toluene (2.25 eq; CA18-0119), Cesium fluoride (3.0 eq; F17-04152) and methanol (1.0 eq) were added. A mixture of water (0.5 eq) and acetonitrile (0.5 eq) was prepared. ¼ of the prepared ACN solution was added to the reaction mixture that was subsequently stirred for 1 h at Ti=20° C. The second portion ACN solution was added and the mixture stirred for another hour. This was repeated two more times. After addition of the last ACN/water-portion the reaction mixture was stirred 180 min at Ti=20° C.

The mixture was quenched by addition of 7.4% NaHCO₃ aq (4 vol) and was stirred for 50 min at Ti=20° C. The biphasic mixture was filtered over a celite bed (2 wt; conditioned upfront with 12 vol toluene). The filter cake was rinsed with toluene (3 vol). The phases were separated and the aqueous layer was extracted with toluene (3 vol). The united organic layers were washed with half sat. NaHCO₃ aq. (5 vol). The organic layer was dried over Na₂SO₄ (2.0 wt), the Na₂SO₄ filteredand the filter cake rinsed with toluene (2 vol). 4-Methylmorpholine (1.0 eq; F17-03830) was added to the product solution. The solution was stored overnight at 4° C.

Step 9

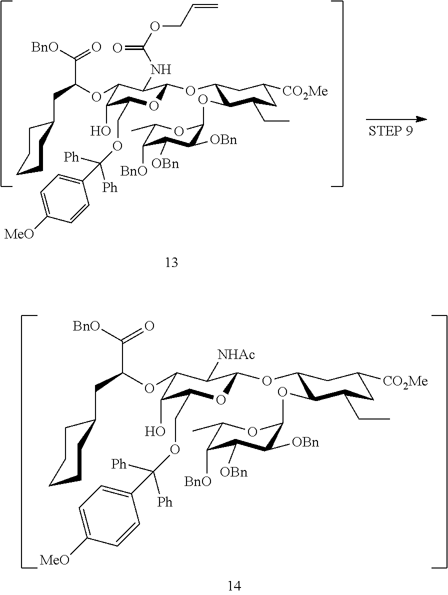

Compound 14: The organic phase comprising Compound 13 was concentrated to 5 vol on the rotavap at Ta=55° C./200-90 mbar. 4-Methylmorpholine (20 eq) and DCM (8 vol) were charged. Acetic anhydride (8 eq) and acetic acid (2 eq; F16-04758) were added at Ti=20° C. The flask was evacuated and purged with nitrogen three times. Triphenylphosphine (0.05 eq) and Pd[(C6H5);P]+(0.05 eq) were added followed by another evacuation/nitrogen purge cycle. The reaction mixture was stirred for 18 h at Ti=20° C.

The reaction was quenched by addition of water (5 vol) over 20 min at ambient temperature. The phases were separated and the organic layer was washed with citric acid 15% w/w aq. (5 vol). The organic phase was charged with sat. NaHCO₃ (5 vol) and methanol (0.5 vol). The mixture was vigorously stirred for 45 min at ambient temperature. The phases were separated and the organic phase was washed twice with water (each time 5 vol) and concentrated on the rotavap to 7 vol at Tj=50° C./600 mbar.

Step 10

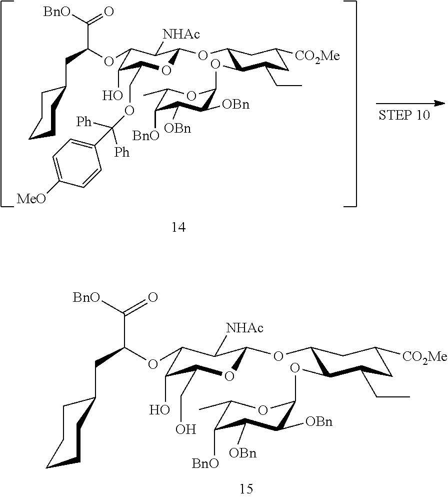

Compound 15: The concentrate (140 mL) comprising Compound 14 was charged with methanol (0.2 vol) and water (0.5 vol) and cooled to Ti=0-5° C. A mixture of TCA (3.0 eq) and DCM (1 vol) was prepared and dosed to the concentrate over 20 min at Ti=1-2° C. The reaction mixture was stirred at this temperature for 3.5 h.

Sat. NaHCO₃ aq. (5 vol) was dosed to the reaction mixture at Ti=1-3° C. within 25 min and the mixture was allowed to warm up to room temperature. The phases were separated and the aqueous phase was extracted with DCM (2 vol). The united organic layers were washed with water (5 vol) and dried over Na₂SO₄ (1.5 wt). The Na₂SO₂ was filtered and rinsed with DCM (2 vol).

Purification: A chromatography column was charged with 1548 g (10 wts) silica gel (15 cm diameter, bed height 22 cm) and conditioned with ethyl acetate/heptanes 1:1. 582 g product solution from step 6/7/8 telescope (starting material: 157.63 g) was charged on top of the column and pre-eluted with 15 ml of DCM. The column was eluted at first applying 60 vol (9.5 L) of eluent 1 (ethyl acetate/heptanes 1:1: after collecting 1 L of wash fractions 19 fractions 1 #1 to 1 #19 (0.5 L vol each) were collected. Aftertwards the eluent was changed to eluent 2 (ethyl acetate/heptanes 3:1), collecting further fractions 1 #20 to 1 #33 (1.0 L vol each). Fractions were analyzed by TLC: pool 1: fractions 1 #18 to 1 #29 were pooled and concentrated furnishing Compound 15 as 80.88 g solid residue, 98.15% a/a. Fractions 1 #15 to 1 #17 were collected as second pool II furnishing a second crop Compound 15 as 9.98 g solid residue, 67.1% a/a.

¹H NMR (Chloroform-d) δ 7.20-7.45 (m, 24H), 5.66 (d. J=6.8 Hz, 1H), 5.14-5.25 (m, 2H), 5.05 (d, J=8.4 Hz, 1H), 4.69-5.01 (m, 7H), 4.61 (d, J=11.4 Hz, IH), 4.35 (dd, J=10.6, 3.0 Hz, 1H), 3.95-4.12 (m, 3H), 3.76-3.87 (m, 2H), 3.59-3.74 (m, 7H), 3.41 (t, J=4.7 Hz, 1H), 3.29 (t, J=9.6 Hz, 1H), 3.08-3.21 (m, 1H), 2.66 (dd, J=9.5, 2.2 Hz, 1H), 2.29 (tt, J=12.6, 3.1 Hz, 1H), 2.13 (d, J=12.7 Hz, 1H), 1.91-2.08 (m, 5H), 1.36-1.81 (m, 13H), 0.99-1.31 (m, 9H), 0.72-0.98 (m, 5H). MS: Calculated for C₆₁H₇₉NO₁₅=1066.28, Found m/z=1088.5 (M+Na).

### Step 11

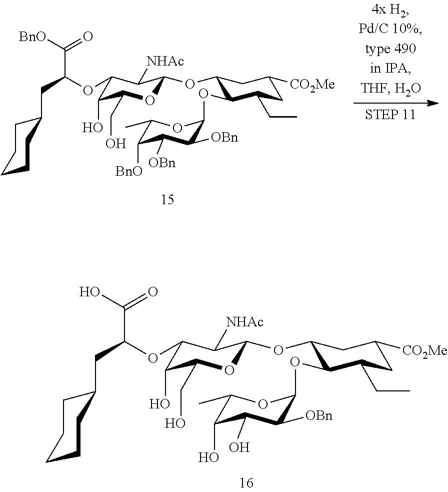

Compound 16: Compound 15 (5.03 g; 1 wt; CA18-0480) was charged with 2-propanol (15 vol), water 0.5 vol) and THF (2.5 vol). The suspension was warmed to Ti=30° C. to obtain a solution. Pd/C 10% 0.2 wt; F15-01378) and 2-propanol (3 vol) were added and the mixture was stirred under hydrogen atmosphere at atmospheric pressure and Tj=37° C. for 7 h. Degassed water (1.5 vol) was added to the reaction mixture and hydrogenation was continued at Tj=37° C./1 bar for 17 h. Degassed water (2 vol) was added and the hydrogenation continued above given conditions for another 7 h. The reaction mixture was stirred overnight under hydrogen atmosphere at Tj=37° C./1 bar.

The hydrogen atmosphere was exchanged for nitrogen and solid NaHCO₃ (0.05 eq) and water (2 vol) were charged. The reaction mixture was filtered at 30° C. over a 0.45 μm nylon membrane and the filter cake was rinsed with a mixture of 2-propanol (3 vol) and water (1 vol). The combined filtrates were concentrated to dryness at Tj=35° C./vac resulting in 4.80 g of solid material. The solid was dissolved in a mixture of water (0.2 vol) and THF (3 vol) to give a clear solution.

Isopropylacetate (25.5 vol) was cooled to Ti=0° C. and the product solution added via dropping funnel over 55 min at Ti=0° C. The dropping funnel was rinsed with a mixture of water (0.1 vol) and THF (0.3 vol). The suspension was filtered after being stirred for 80 min at Ti=0° C. The filter cake was rinsed with MTBE (3 vol) and the product was dried under vacuum and nitrogen flow overnight. 3.10 g n.corr./3.08 g LoD corr. (Y LoD corr 92.66%).

¹H NMR (400 MHZ, DMSO-d6) δ 4.61-4.83 (m, 2H), 4.08-4.26 (m, 3H), 3.98 (d, J=8.6 Hz, 1H), 3.80 (s, 1H), 3.29-3.57 (m, 10H), 3.19-3.28 (m, 1H), 3.06 (t, J=9.5 Hz, 1H), 2.34-2.47 (m, 1H), 2.22 (d, J=12.7 Hz, 1H), 1.91-2.04 (m, 1H), 1.71-1.89 (m, 5H), 1.34-1.69 (m, 8H), 0.68-1.31 (m, 13H). MS: Calculated for C₃₃H₅₅NO₁₅=705.79, Found m/z=728.4 (M+Na).

1. A process for making Compound 16 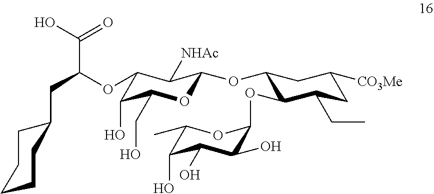
wherein said process comprises at least one step chosen from:
(a) epoxide opening of Compound 2a 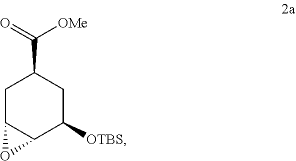 [SMILES: COC(=O)[C@@H]1C[C@H]2O[C@H]2[C@H](O[Si](C)(C)C(C)(C)C)C1]
wherein the epoxide opening of Compound 2a comprises the use of at least one ethyl magnesium halide, at least one copper(I) salt, and at least one lewis acid; and
(b) epoxidation of Compound 1 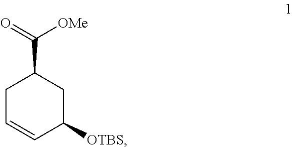 [SMILES: COC(=O)[C@@H]1CC=C[C@H](O[Si](C)(C)C(C)(C)C)C1]
wherein the epoxidation of Compound 1 comprises the use of potassium peroxymonosulfate and at least one base.

2. The process according to claim 1, wherein the process comprises steps (a) and (b).

3. The process according to claim 1, wherein the at least one ethyl magnesium halide is ethyl magnesium chloride.

4. The process according to claim 1, wherein the at least one copper(I) salt is copper(I) bromide-dimethyl sulfide complex.

5. The process according to claim 1, wherein the at least one lewis acid is boron trifluoride etherate.

6. The process according to claim 1, wherein the at least one base is chosen from metal carbonates.

7. The process according to claim 6, wherein the metal carbonate is NaHCO₃.

8. The process according to claim 1, wherein step (a) is performed in the presence of THF and/or cyclopentylmethyl ether.

9. The process according to claim 8, wherein step (a) is performed in the presence of THF.

10. The process according to claim 8, wherein step (a) is performed in the presence of cyclopentylmethyl ether.

11. The process according to claim 1, wherein step (a) is performed at a temperature within the range of −100° C. to −60° C.

12. The process according to claim 11, wherein step (a) is performed at about −78° C.

13. The process according to claim 1, wherein the molar ratio of the copper(I) salt to the ethyl magnesium halide in step (a) is about 1 to 3.

14. The process according to claim 1, wherein step (b) is performed in the presence of acetone and/or water.

15. The process according to claim 14, wherein step (b) is performed in the presence of acetone and water.

16. The process according to claim 1, wherein step (b) is performed at a temperature within the range of −10° C. to 30° C.

17. The process according to claim 16, wherein step (b) is performed at about 0° C.

18. A process for making Compound 3 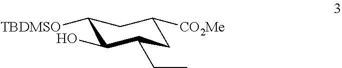
wherein said process comprises at least one step chosen from:
(a) epoxide opening of Compound 2a 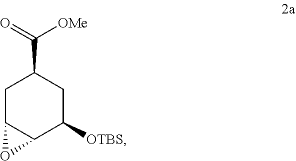 [SMILES: COC(=O)[C@@H]1C[C@H]2O[C@H]2[C@H](O[Si](C)(C)C(C)(C)C)C1]
wherein the epoxide opening of Compound 2a comprises the use of at least one ethyl magnesium halide, at least one copper(I) salt, and at least one lewis acid; and
(b) epoxidation of Compound 1 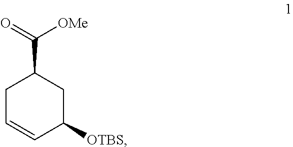 [SMILES: COC(=O)[C@@H]1CC=C[C@H](O[Si](C)(C)C(C)(C)C)C1]
wherein the epoxidation of Compound 1 comprises the use of potassium peroxymonosulfate and at least one base.

19. The process according to claim 18, wherein the process comprises steps (a) and (b).

20. The process according to claim 18, wherein the at least one ethyl magnesium halide is ethyl magnesium chloride.

21. The process according to claim 18, wherein the at least one copper(I) salt is copper(I) bromide-dimethyl sulfide complex.

22. The process according to claim 18, wherein the at least one lewis acid is boron trifluoride etherate.

23. The process according to claim 18, wherein the at least one base is chosen from metal carbonates.

24. The process according to claim 23, wherein the metal carbonate is NaHCO₃.

25. The process according to claim 18, wherein step (a) is performed in the presence of THF and/or cyclopentylmethyl ether.

26. The process according to claim 25, wherein step (a) is performed in the presence of THF.

27. The process according to claim 25, wherein step (a) is performed in the presence of cyclopentylmethyl ether.

28. The process according to claim 18, wherein step (a) is performed at a temperature within the range of −100° C. to −60° C.

29. The process according to claim 28, wherein step (a) is performed at about −78° C.

30. The process according to claim 18, wherein the molar ratio of the copper(I) salt to the ethyl magnesium halide in step (a) is about 1 to 3.

31. The process according to claim 18, wherein step (b) is performed in the presence of acetone and/or water.

32. The process according to claim 31, wherein step (b) is performed in the presence of acetone and water.

33. The process according to claim 18, wherein step (b) is performed at a temperature within the range of −10° C. to 30° C.

34. The process according to claim 33, wherein step (b) is performed at about 0° C.

35. A process for making Compound 16 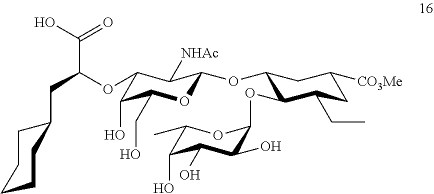
wherein said process comprises a step of epoxide opening of Compound 2a 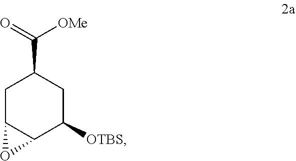 [SMILES: COC(=O)[C@@H]1C[C@H]2O[C@H]2[C@H](O[Si](C)(C)C(C)(C)C)C1]
wherein the epoxide opening of Compound 2a comprises the use of at least one ethyl magnesium halide, at least one copper(I) salt, and at least one lewis acid.

36. The process according to claim 35, wherein the at least one ethyl magnesium halide is ethyl magnesium chloride.

37. The process according to claim 35, wherein the at least one copper(I) salt is chosen from copper(I) halides, copper(I) triflates, copper(I) thiophenoxide, copper(I) cyanide, and 2-thienyl(cyano)copper lithium.

38. The process according to claim 35, wherein the at least one copper(I) salt is copper(I) chloride.

39. The process according to claim 35, wherein the at least one copper(I) salt is copper(I) bromide.

40. The process according to claim 35, wherein the at least one copper(I) salt is copper(I) iodide.

41. The process according to claim 35, wherein the at least one copper(I) salt is copper(I) bromide-dimethyl sulfide complex.

42. The process according to claim 35, wherein the at least one lewis acid is chosen from boron trihalides and aluminum triflate.

43. The process according to claim 35, wherein the at least one lewis acid is chosen from boron trifluoride, boron trichloride, and boron tribromide.

44. The process according to claim 35, wherein the at least one lewis acid is boron trichloride.

45. The process according to claim 35, wherein the at least one lewis acid is boron tribromide.

46. The process according to claim 35, wherein the at least one lewis acid is boron trifluoride.

47. The process according to claim 35, wherein the at least one lewis acid is boron trifluoride etherate.

48. The process according to claim 35, wherein the step of epoxide opening is performed in the presence of THF and/or cyclopentylmethyl ether.

49. The process according to claim 48, wherein the step of epoxide opening is performed in the presence of THF.

50. The process according to claim 48, wherein the step of epoxide opening is performed in the presence of cyclopentylmethyl ether.

51. The process according to claim 35, wherein the step of epoxide opening is performed at a temperature within the range of −100° C. to −60° C.

52. The process according to claim 51, wherein the step of epoxide opening is performed at about −78° C.

53. The process according to claim 35, wherein the molar ratio of the copper(I) salt to the ethyl magnesium halide is about 1 to 3.

54. A process for making Compound 3 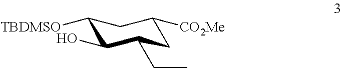
wherein said process comprises a step of epoxide opening of Compound 2a 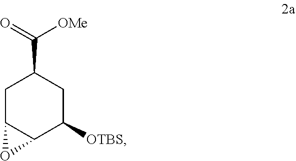 [SMILES: COC(=O)[C@@H]1C[C@H]2O[C@H]2[C@H](O[Si](C)(C)C(C)(C)C)C1]
wherein the epoxide opening of Compound 2a comprises the use of at least one ethyl magnesium halide, at least one copper(I) salt, and at least one lewis acid.

55. The process according to claim 54, wherein the at least one ethyl magnesium halide is ethyl magnesium chloride.

56. The process according to claim 54, wherein the at least one copper(I) salt is chosen from copper(I) halides, copper(I) triflates, copper(I) thiophenoxide, copper(I) cyanide, and 2-thienyl(cyano)copper lithium.

57. The process according to claim 54, wherein the at least one copper(I) salt is copper(I) chloride.

58. The process according to claim 54, wherein the at least one copper(I) salt is copper(I) bromide.

59. The process according to claim 54, wherein the at least one copper(I) salt is copper(I) iodide.

60. The process according to claim 54, wherein the at least one copper(I) salt is copper(I) bromide-dimethyl sulfide complex.

61. The process according to claim 54, wherein the at least one lewis acid is chosen from boron trihalides and aluminum triflate.

62. The process according to claim 54, wherein the at least one lewis acid is chosen from boron trifluoride, boron trichloride, and boron tribromide.

63. The process according to claim 54, wherein the at least one lewis acid is boron trichloride.

64. The process according to claim 54, wherein the at least one lewis acid is boron tribromide.

65. The process according to claim 54, wherein the at least one lewis acid is boron trifluoride.

66. The process according to claim 54, wherein the at least one lewis acid is boron trifluoride etherate.

67. The process according to claim 54, wherein the step of epoxide opening is performed in the presence of THF and/or cyclopentylmethyl ether.

68. The process according to claim 67, wherein the step of epoxide opening is performed in the presence of THF.

69. The process according to claim 67, wherein the step of epoxide opening is performed in the presence of cyclopentylmethyl ether.

70. The process according to claim 54, wherein the step of epoxide opening is performed at a temperature within the range of −100° C. to −60° C.

71. The process according to claim 70, wherein the step of epoxide opening is performed at about −78° C.

72. The process according to claim 54, wherein the molar ratio of the copper(I) salt to the ethyl magnesium halide is about 1 to 3.
